(19) United States
(12) Patent Application Publication
(10) Pub. No.: US 2014/0073598 A1
(43) Pub. Date: Mar. 13, 2014

(54) MULTIPLE FOLATE FORMULATION AND USE THEREOF

(71) Applicants: Rhett Sean Daniels, Encinitas, CA (US); Harold Robinson, Sunset, LA (US)

(72) Inventors: Rhett Sean Daniels, Encinitas, CA (US); Harold Robinson, Sunset, LA (US)

(73) Assignee: JAYMAC PHARMACEUTICALS LLC, Sunset, LA (US)

(21) Appl. No.: 13/609,985

(22) Filed: Sep. 11, 2012

(51) Int. Cl. A61K 31/714; A61P 25/24

(52) U.S. Cl. 514/52

(57) ABSTRACT

A multiple folate composition having at least 3 different forms of folate comprising at least one of folic acid (a salt or ester thereof); at least one of a folinic acid (a salt or ester thereof); and at least one of a 5-methyl-tetrahydrofolic acid (a salt or ester thereof) is disclosed. The composition is useful as a nutritional supplement or medication in the treatment of folate deficiency and the sequella thereof and/or in conditions responsive to administration of metabolically useful folate. The compositions are particularly of use in patients who have impaired or reduced ability to convert folic acid to its metabolically active forms, and in the treatment of depression.

### FIELD OF INVENTION

The invention, is in the field of nutritional supplementation using folates and medicinal uses thereof in the treatment or prevention of various conditions related to such supplementation, including, but not limited to depression.

### BACKGROUND

Folate is an essential nutrient in humans and other animals. The human body needs folate to synthesize DNA, repair DNA, and methylate DNA as well as to act as a cofactor in biological reactions involving folate. It is especially important in aiding rapid cell division and growth, such as in infancy and pregnancy. Children and adults both require folic acid to produce healthy red blood cells and prevent anemia.

Generally, in the body, folic acid is converted to dihydrofolic acid, which can be converted to tetrahydrofolic acid or 5,10-methylene tetrahydrofolic acid. Tetrahydrofolic acid and 5, 10-methylenetetralydrofolic acid can also be converted back to dihydrofolic acid or converted to one another (a) directly or (b) each can be converted to 5-methylhydrofolic acid as an intermediary before conversion to the other. The structural changes can be seen more particularly in FIG. 1. The most abundant form in natural foods such as green leafy vegetables, legumes, liver, and egg yolk is 5-methyltetrahydrofolate (MTHF), while the usual dietary supplement form is folic acid (or a salt or ester thereof). Unfortunately, the active compound for use in the body is not folic acid per se, and a significant portion of the population has one or more defects in the metabolic pathways leading to the active form so that such individuals do not get the full benefit or (in severe cases) any benefit from folic acid supplementation. In this specification, unless specified otherwise or the context requires otherwise, the term “folate” will be used to refer to all forms of folic acid and its normal metabolically useful metabolites as well as materials that the body can usually convert into folic acid and/or metabolically useful folic acid metabolites.

Folate deficiency can result in many health problems, the most notable one being neural tube defects in developing embryos. Common symptoms of folate deficiency include diarrhea, macrocytic anemia with weakness or shortness of breath, nerve damage with weakness and limb numbness (peripheral neuropathy), pregnancy complications, mental confusion, forgetfulness or other cognitive declines, mental depression, sore or swollen tongue, peptic or mouth ulcers, headaches, heart palpitations, irritability, and behavioral disorders. Low levels of folate can also lead to homocysteine accumulation. DNA synthesis and repair are impaired and this could lead to cancer development.

Adequate folate intake during the periconception period, the time right before and just after a woman becomes pregnant, helps protect against a number of congenital defects, including neural tube defects (which are the most notable birth defects that occur from folate deficiency). Neural tube defects produce malformations of the spine, skull, and brain including spina bifida and anencephaly. The risk of neural tube defects is significantly reduced when supplemental folic acid is consumed in addition to a healthy diet prior to and during the first month following conception. Supplementation with folic acid has also been shown to reduce the risk of congenital heart defects, cleft lips, limb defects, and urinary tract anomalies. Folate deficiency during pregnancy may also increase the risk of preterm delivery, infant low birth weight and fetal growth retardation, as well as increasing homocysteine level in the blood, which may lead to spontaneous abortion and pregnancy complications, such as placental abruption and pre-eclampsia. Women who could become pregnant are advised to eat foods fortified with folic acid or take supplements in addition to eating folate-rich foods to reduce the risk of serious birth defects. The mechanisms and reasons why folic acid prevents birth defects is unknown, however it has been hypothesized that the insulin-like growth factor 2 (IGF2) gene is differentially methylated and these changes in IGF2 result in improved intrauterine growth and development.

Folic acid may also reduce chromosomal defects in sperm. Folate is necessary for fertility in both men and women. In men, it contributes to spermatogenesis. In women, on the other hand, it contributes to oocyte maturation, implantation, and placentation, in addition to the general effects of folic acid on pregnancy. Therefore, it is necessary to receive sufficient amounts through the diet to avoid subfertility.

Folic acid appears to reduce the risk of stroke. The literature indicates the risk of stroke appears to be reduced only in some individuals, but a definite recommendation regarding supplementation beyond the current RDA has not been established for stroke prevention. Observed stroke reduction is consistent with the reduction in pulse pressure produced by folate supplementation of 5 mg per day, since hypertension is a key risk factor for stroke.

Some evidence links a shortage of folate with depression, and that folate supplementation can treat it, either when used alone or in conjunction with antidepressants, especially the selective serotonin reuptake inhibitors (SSRIs). Under proper conditions, folic acid supplementation has been shown to affect noradrenaline and serotonin receptors within the brain which could be the cause of folic acid's possible ability to act as an antidepressant.

Depression is a debilitating condition estimated to affect more than 21 million Americans, and is a leading cause of disability in developed economies. It is often recurrent, and can range from mild to severe, with symptoms of sadness, hopelessness, fatigue, anxiety, and difficulty concentrating, loss of appetite, among others. In its severe manifestations, depression can be deadly.

While the administration of antidepressant medications is the most common treatment for depression, the drawbacks of these drugs are widely recognized. Standard antidepressants are commonly ineffective, or only partially effective, and not all patients respond in the same way to the medications. In addition, these drugs often have distressing side effects, including weight gain, sexual dysfunction, and sleep disturbances. A most disturbing side effect in a number of antidepressant medications, especially those of the selective serotonin reductase inhibitor (SSRI) class is the increased risk of suicidal thoughts and the potential for acting on such thoughts. Side effects, among other issues, can lead to diminished patient compliance/adherence. In one 1995 study, for example, 28% of patients stopped taking their antidepressant medication the first month of therapy, and by the third month, 44% had stopped taking it.

Folate deficiency may increase the risk of schizophrenia because, by increasing homocysteine levels, folate also increases interleukin 6 and tumor necrosis factor alpha levels, and these two cytokines are involved in the development of schizophrenia. The exact mechanisms involved in the development of schizophrenia are not entirely clear, but may have something to do with DNA methylation and one carbon metabolism, and these are the precise roles of folate in the body.

Nutritional supplement containing folic acid, pyridoxine and cyanocobalamin decreased the risk of developing age related macular degeneration.

Folate deficiency may lead to glossitis, diarrhea, depression, confusion, anemia, and fetal neural tube defects and brain defects (during pregnancy). Folate deficiency is accelerated by alcohol consumption. Folate deficiency has been treated with supplemental folic acid (or salt or ester thereof) of 400 to 1000 μg per day. This treatment is very successful in replenishing tissues, even if deficiency was caused by malabsorption.

All the biological functions of folic acid are performed by tetrahydrofolate and other derivatives. Their biological availability to the body depends upon dihydrofolate reductase action in the liver. This action is unusually slow in humans, being less than 2% of that in rats. Moreover, in contrast to rats, an almost-5-fold variation in the activity of this enzyme exists between humans. Due to this low activity, it has been suggested that this limits the conversion of folic acid into its biologically active forms “when folic acid is consumed at levels higher than the Tolerable Upper Intake Level (1 mg/d for adults).” Also, polymorphisms in genes of enzymes involved in folate metabolism results in some patients having deficiencies in metabolically useful folates despite having what would appear to be normal or greater than normal intake of folic acid (and/or its salts and/or esters).

Homocysteine is a sulfur-containing amino acid produced by the biosynthesis of the essential amino acid methionine. Elevated levels of homocysteine have been demonstrated to be a risk factor for cardiovascular diseases, as well as many other conditions. Folate is intimately involved in regulating homocysteine levels via the homocysteine remethylation and transsulfuration metabolic pathways. (See FIG. 2.) These pathways require certain B vitamin co-factors along with productive enzymatic processes to function effectively importantly, then, nutritional deficiencies and genetic polymorphisms can compromise these critical metabolic processes.

The remethylation pathway recycles homocysteine back to methionine, an essential amino acid, via the cobalamin (B₁₂)-dependent methionine synthase (MS) enzyme. Methionine synthase also requires an active, reduced form of folate known as 5-methyltetrahydrofolate (MTHF). This is the main form of folate that occurs naturally in foods (with different number of glutamates), such as in green leafy vegetables, legumes, liver, and egg yolk. However, in folic acid-fortified foods and supplements, the form of folate ingested must undergo conversion via dihydrofolate reductase to tetrahydrofolate. The methlyenetetrahydrofolate reductase (MTHFR) enzyme then plays a role, reducing 5,10 methylenetetrahydrofolate to MTHF, which participates in methionine synthase reactions. Notably, remethylation produces the ubiquitous methyl donor S-adenosyl-L-methionine (SAM) as an intermediate step. SAM acts as methyl group donating-cofactor for tryptophan and tyrosine hydroxylase activities involved in the synthesis of brain neurotransmitters serotonin, dopamine, and norepinephrine, (See FIGS. 3 and 4)

The transsulfuration pathway irreversibly converts homocysteine to cysteine, a non-essential amino acid (the body can generally synthesize it, however, since some populations cannot, it is considered a “conditionally essential amino acid). In the process, cystathionine, a key intermediate, is synthesized via an “upper” reaction of cystathionine-beta-synthase (CBS) and serine. In a “lower” reaction, cystathione gamma lyase converts cystathionine to cysteine. Both of these reactions require a vitamin B₆ co-factor, namely pyridoxal phosphate (PLP). Without an adequate supply of PLP, the conversion cannot proceed effectively, leading to higher concentrations of homocysteine and lower concentrations of cysteine. In addition, because cysteine yields the neuromodulator, hydrogen sulfide, decreased cysteine correlates with reduced hydrogen sulfide. Studies have reported that brain hydrogen sulfide is diminished in Alzheimer's disease patients, which intimates that this neuroamodulator may play a role in cognitive decline.

Polymorphisms of the genes encoding key enzymes in the methionine cycle are not rare. For example, genetic variants of the methylenetrahydrofolate reductase (MTHFR) enzyme commonly occur in up to 50% of the population. Notably, these are present with more frequency among depression sufferers. One of the more well-studied variants is C677T, which, in its homozygous state, results in 50% decreased, enzymatic activity. Individuals who possess a MTHFR genetic mutation cannot efficiently reduce 5,10-methylenetetrahydrofolate to the active MTHF (i.e. 5-methyltetrahydrofolate), which is a necessary step in homocysteine remethylation, it is not surprising, then, that hyperhomocysteinemia and increased risk for development of folate-mediated Neural Tube Defects (NTDs) and vascular disease are associated with this genotype.

There are numerous causes of hyperhomocysteinemia. Reduced intake of dietary folate and incomplete/impaired dihydrofolate reduction (e.g., due to genetic polymorphisms, conditions affecting the liver, such as alcoholism) are just two of those. Additionally, those with poor kidney functioning, and malabsorptive conditions, such as celiac disease, are also at risk for hyperhomocysteinemia. Studies have also shown that the administration of certain medications, including antifolates, such as methotrexate, suppresses the homocysteine remethylation cycle by blocking necessary enzymatic activities. Proton pump inhibitors, H2 blockers, metformin, anticonvulsants, and many other medications can affect folate absorption and homocysteine metabolism.

Maternal folate deficiency and hyperhomocysteinemia are recognized as risk factors for the development of NTDs in offspring. Numerous studies suggest that nutritional status and amino acid metabolism play a role in depression, too. The Rotterdam study, for example, demonstrated an association between depression and folate, vitamin B₁₂, and homocysteine. While the exact mechanisms are not fully understood, it is theorized that nutrient deficiencies disturb the methionine cycle, affecting SAM and tetrahydrobiopterin (BH4) production, which are needed for the synthesis of the three mood-regulating monoamine neurotransmitters: dopamine, norepinephrine, and serotonin. This impaired synthesis of dopamine, norepinephrine, and serotonin leads to depression.

The question arises, however, as to whether nutritional deficiencies cause the depression or whether they are a consequence of one of the decreased appetite and self-neglect (or a combination of both) that is seen in many depressed individuals. Either way, the studies suggest that supplementation as a means to improving homocysteine metabolism is a viable therapeutic option for depressed patients.

Various other nutritional supplement entities participate in the metabolic cycles in which folates are active, especially in the homocysteine metabolic pathways. One such material is pyridoxal 5′ phosphate (PLP) is the active form of pyridoxine. It is a cofactor for cystathionine beta synthase, which is an integral enzyme in the transsulfuration pathway.

The cobalamin derivatives methylcobalamin and adenosylcobalamin are endogenous forms of vitamin B₁₂. Reduced, active forms of cobalamin ensure adequate supplementation even in the presence of polymorphisms that limit their synthesis. Methylcobalamin is a cofactor for cobalamin-dependent methionine synthase, which recycles homocysteine back to methionine. Without the B₁₂ cofactor, the MTHF gets caught in the “methyl group trap” and it can participate in neither remethylation nor regeneration of tetrahydrofolate. Adenosylcobalamin is required for methylmalonyl-CoA mutase, which isomerizes methylmalonyl-CoA to succinyl-CoA. Succinyl-CoA is integral to the synthesis of hemoglobin. In addition, a deficiency of adenosylcobalamin results in an accumulation of methylmalonic acid, which has been shown to be associated with depression.

It is therefore an object of the invention to provide a folate supplementation formulation that addresses the inability to fully convert folic acid in vivo to its active metabolites.

It is another object of the invention to provide intermediary and fully active forms of folic acid (i.e. tetrahydrofolates) as a nutritional supplementation.

It is yet another object of the invention to treat or prevent conditions associated with folate deficiency with the above nutritional supplement.

It is still a further object of the invention to treat or prevent or reduce the severity of conditions that are responsive to various forms of folate supplementation regardless of whether the patient has a folate deficiency and regardless of whether the patient can fully metabolize folic acid to its active form.

It is still another object of the invention to provide a nutritional supplement that reduces homocysteine accumulation in patients via multiple supplementation approaches that avoid the need for metabolic transformation of the many of the supplements into active forms.

Still another object of the invention is to provide a treatment of depression that is associated with at least one of folate deficiency and/or homocysteine accumulation.

Yet other objects of the invention are one or more of (a) to reduce the rate of cognitive decline; and (b) increase mental alertness, that are related to effective metabolically useful folate deficiency and/or homocysteine accumulation.

Still other objects of the invention will be apparent to those of ordinary skill in the art.

### SUMMARY OF THE INVENTION

The present invention is a folate-B12 composition comprising

(1) at least 3 forms of folate selected from the group comprising
a) at least one of folic acid (aka pteroylmonoglutamic acid), a pharmaceutically acceptable salt thereof, a pharmaceutically acceptable ester thereof, and mixtures thereof;
b) at least one of a folinic acid (aka formyltetrahydrofolate, aka formylTHF) comprising at least 5-formyl-tetrahydro folic acid (preferably a diasterioisomerically enriched (6S) form of 5-formyl-tetrahydofolic acid), a pharmaceutically acceptable salt thereof, a pharmaceutically acceptable ester thereof, and mixtures thereof; and
c) at least one of a 5-methyl-tetrahydrofolic acid (aka 1-methylfolate, aka MeTHF, aka MTHF) (preferably comprising at least a diastereoisomerically enriched 1-methyl-folate), a pharmaceutically acceptable salt thereof, a pharmaceutically acceptable ester thereof, and mixtures thereof;
   (2) at least one vitamin B12 component comprising at least one of (and preferably both ot) adenosylcobalamin and methyl colbalamin; and preferably further comprising at least one vitamin B6 in the form of pyridoxy-5′-phosphate. The invention formulation can be used for supplementation of folate to patients who have a folate deficiency; as a medicinal for patients who, although not having a folate deficiency, would benefit for still higher levels of folate; and for supplementation to patients that have an impaired ability to convert folic acid to its active forms. The invention formulation can also be used for nutritional supplemental control of homocysteine levels in those in need of homocysteine level control. The formulation can also be used in the treatment f depression and other homocysteine and folate imbalance disorders.

### BRIEF DESCRIPTION OF THE DRAWINGS

FIG. 1 shows the metabolic relationship of folic acid and its normal human metabolic products.

FIG. 2 shows the remethylation and transsulfuration patenways and the interplay of homocysteine and folate.

FIG. 3 shows typical serotonin biosynthesis.

FIG. 4 shows typical dopamine biosynthesis.

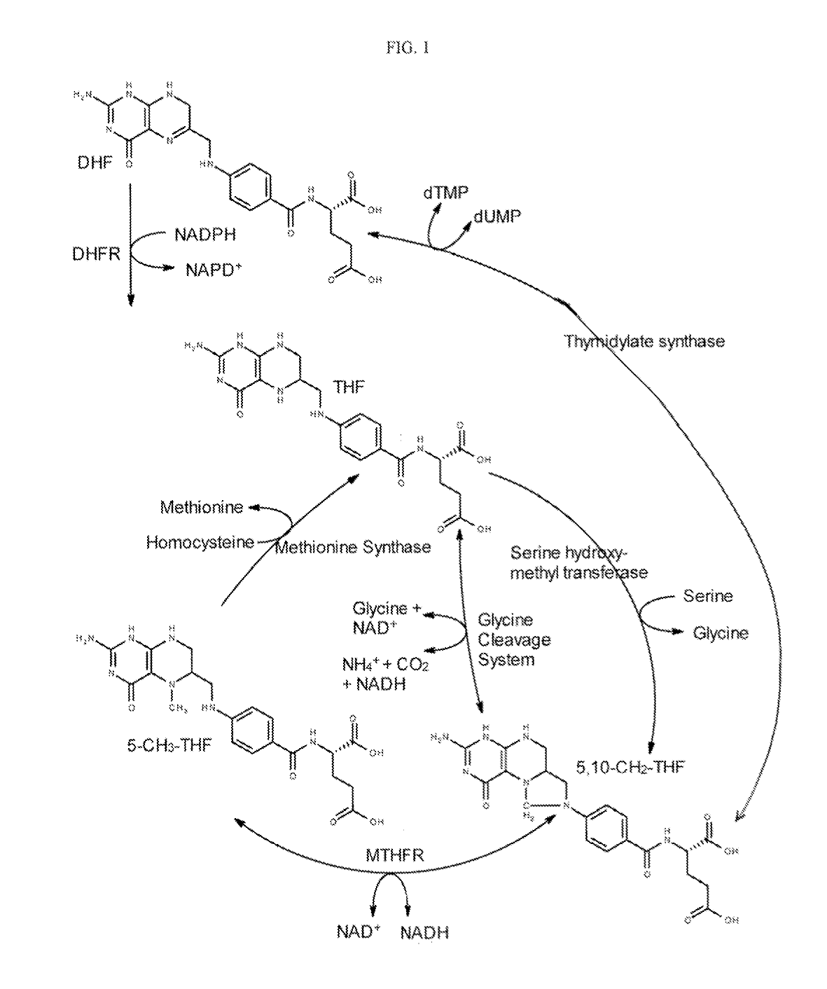

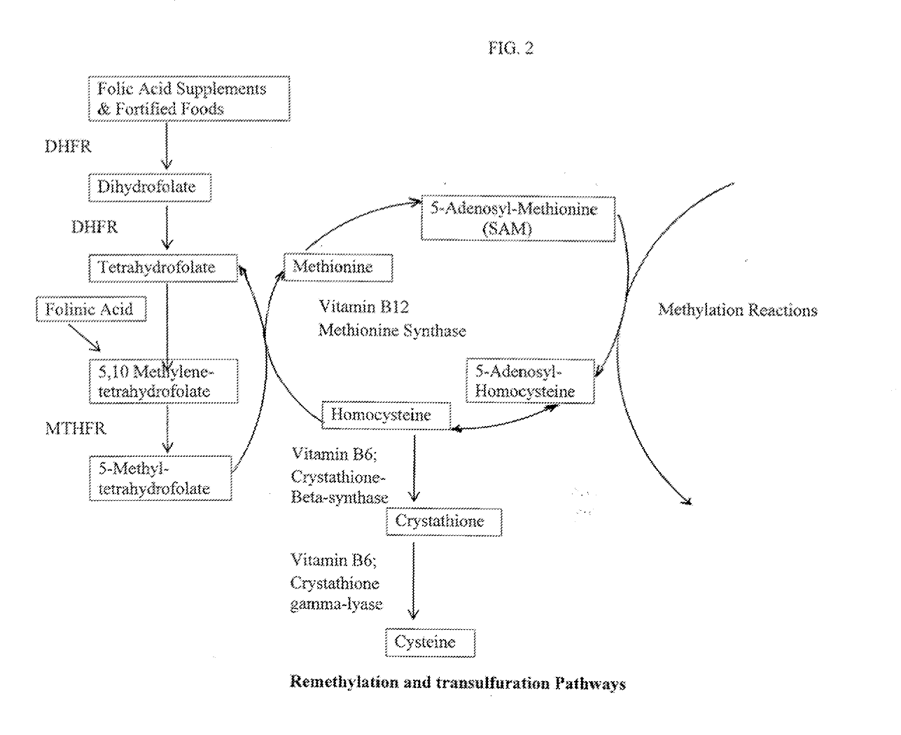

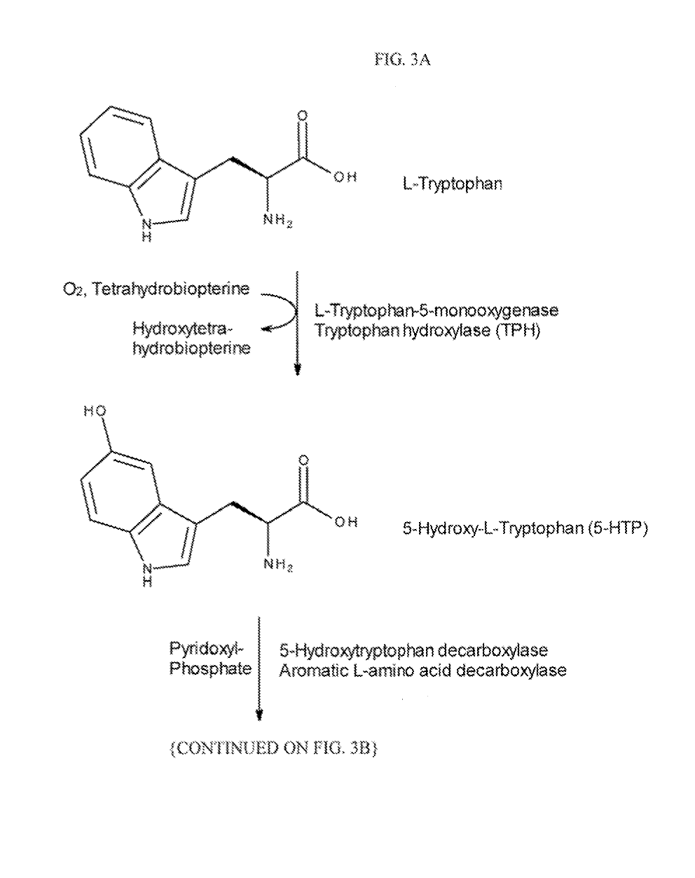

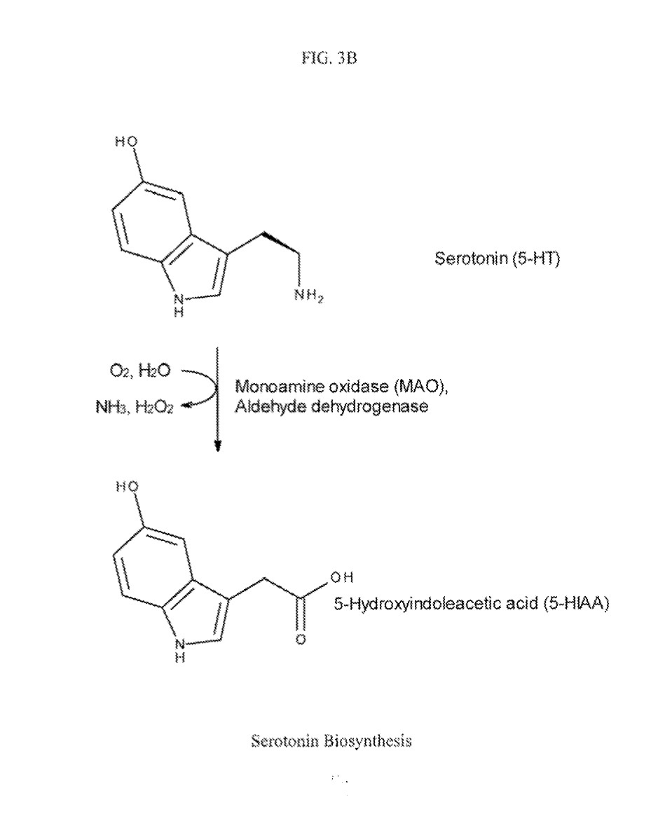

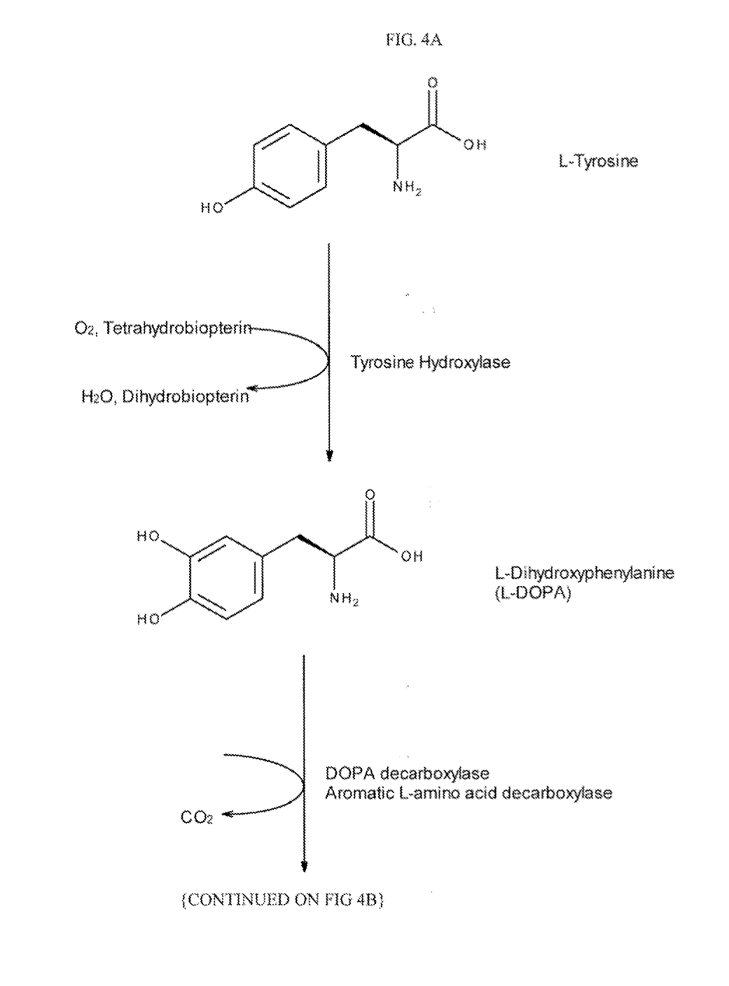

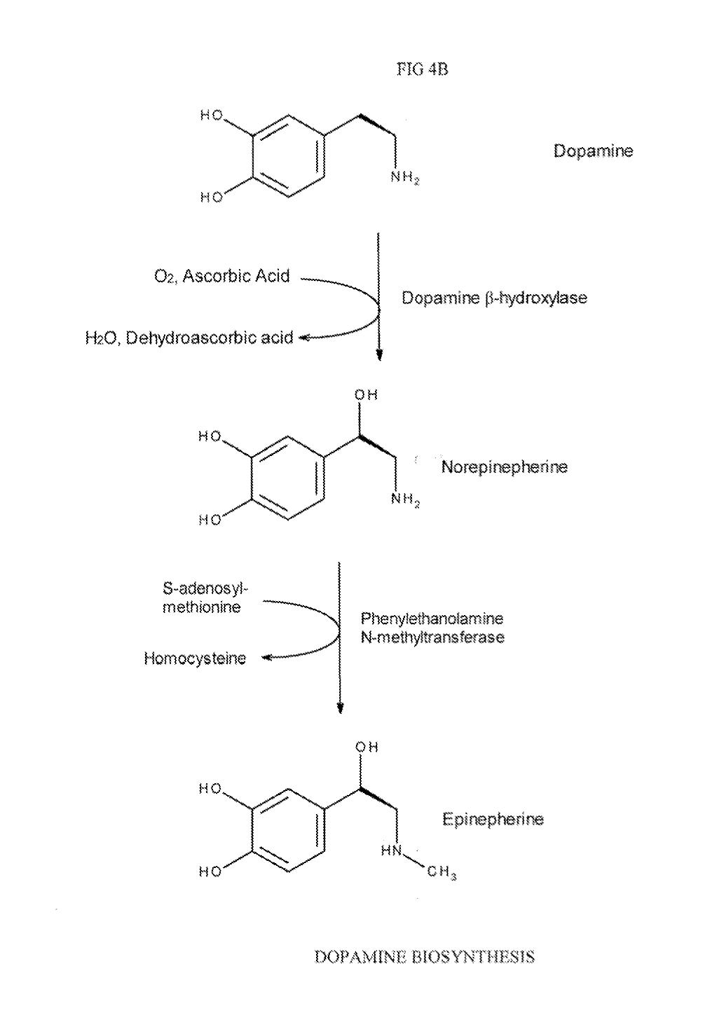

### DETAILED DESCRIPTION OF THE PREFERRED EMBODIMENT

The invention is a folate-B12 composition comprising at least 3 forms of folate in combination with a vitamin B12 component. The 3 forms of folate required are selected from the group comprising
a. at least one of folic acid (aka pteroylmonoglutamic acid), a pharmaceutically acceptable salt thereof, a pharmaceutically acceptable ester thereof, and mixtures thereof;
b. at least one of a folinic acid (aka formyltetrahydrofolate, aka formylTHF) comprising at least a 5-formyl-tetrahydrofolic acid (preferable a diasterioisomerically enriched (6S) form of 5-formyl-tetrahydofolic acid, most preferably a diasteriomerically pure (6S) form of 5-formyl-tetrahydofolic acid), a pharmaceutically acceptable salt thereof, a pharmaceutically acceptable ester thereof, and mixtures thereof; and
c. at least one of a 5-methyl-tetrahydrofolic acid (aka 1-methylfolate, aka MeTHF, aka MTHF) (preferably comprising at least a diastereoisomerically enriched 1-methyl-folate most preferably a diasteriomerically pure 1-methyl-folate (aka 1-5-methyl tetrahydrofolic acid)), a pharmaceutically acceptable salt thereof, a pharmaceutically acceptable ester thereof, and mixtures thereof.
   When used in the present invention, “diasteriomerically pure” is intended to mean at least the referenced material is 98% of the intended isomer, preferably at least 98.5%, more preferably at least 99%, still more preferably at least 99.5%, even more preferably at least 99.9% of the intended isomer. In a preferred embodiment of the invention at least one of said three forms of folate is present as a sugar amine conjugate (such as, without limitation, but preferably, glucosaminyl or galactosaminyl conjugated form thereof, more preferably a D-glucosaminyl or D-galactosaminyl conjugated form thereof). When desired, additional forms of folate may also be present, such as, without limitation, dihydrofolic acid, 5,10-methylene-tetrahydrofolic acid, and/or a tetrahydrofolic acid (other than the foregoing).

The present invention contains three different forms of folate, 1-methylfolate, folinic acid, and folic acid, to increase folate availability as a means to enhance homocysteine degradation. L-methylfolate is the metabolically active form of folate, readily available to participate in homocysteine remethylation reactions. It, unlike folic acid, does not require reduction by MTHFR-dependent process, and is therefore particularly indicated in the presence of a MTHFR polymorphism, such as C677T.

Folinic acid, also known as 5-formyltetrahydrofolate or leucovorin, is a derivative of tetrahydrofolic acid. Widely accepted as a folate rescue strategy to counter methotrexate toxicity in cancer treatment, its reduced folate properties lend itself to folate supplementation, particularly in the presence of dihydrofolate reductase inhibitors, such as pyrimethamine and methotrexate. It is also indicated for patients with certain genotypes of the dihydrofolate reductase enzyme that disturbs the reduction of dihydrofolate to tetrahydrofolate.

Folic acid administration has been shown to lower homocysteine in various populations (without the MTHFR polymorphism).

The vitamin B12 component is at least in the form of one or both of adenosylcobalamin or methylcobalamin, or pharmaceutically acceptable salts, esters, amides, or other metabolically useful prodrugs thereof, preferably in the form of adenosylcobalamin or methylcobalamin. In a preferred embodiment both an adenosylcobalamin (or a pharmaceutically acceptable salt, ester, amide, or other metabolically useful prodrug thereof and a methylcobalamin (or a pharmaceutically acceptable salt, ester, amide, or other metabolically useful prodrug thereof) are present. Methylcobalamin has the structure I below while adenosylcobalamin has the structure II below

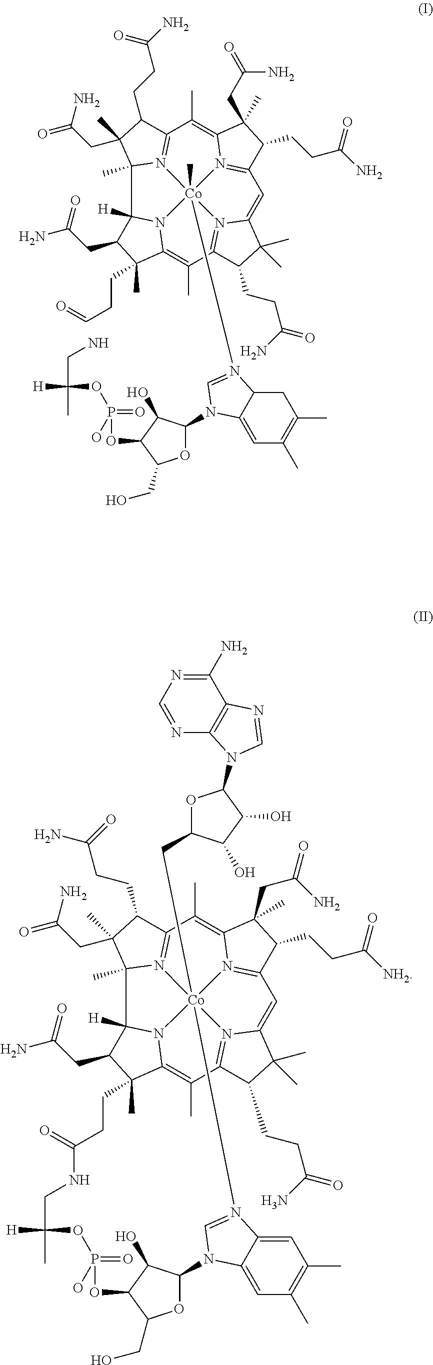

They each differ from cyano cobalamin in the replacement of the CN group (bound to the Co atom) of cyanocobalamin with methyl (methylcobablamine) or adenosyl (adenosylcobalamin), the methyl group and the adenosyl group being bound directly to the Co atom.

In general, the various required folates are present in independent amounts of up to 4 mg each, although they need not be present in equal amount preferably independently up to 3 mg of each, with a preferable minimum of at least 0.4 mcg. In some specific dosage forms preferred dosages include those where the three required forms of foalte are present in equal weights. Other preferred dosage forms contain independently from 0.4 mcg up to 800 mcg of each of the three required forms. In other preferred dosage forms, each of the required three forms of folate is present independently in an amount of at least 2 mg and preferably (but not necessarily) each of these three forms is present in equal weights, in still other preferred dosage forms, each of the three required folate forms is independently present in a range in which the minimum and maximum (with the maximum being greater than the minimum) are selected from 0.4 mcg, 0.8 mcg, 1 mcg, 2 mcg, 5 mcg, 10 mcg, 20 mcg, 25 mcg, 50 mcg, 100 mcg, 200 mcg, 400 mcg, 800 mcg, 1000 mcg, 1200 mcg, 1600 mcg, 2000 mcg, 2400 mcg, 2800 mcg, 3200 mcg, 3600 mcg, and 4000 mcg. In particular embodiments; each of the 3 required folates is independently present in an amount selected from 0.4 mcg, 0.8 mcg, 1 mcg, 2 mcg, 5 mcg, 10 mcg, 20 mcg, 25 mcg, 50 mcg, 100 mcg, 200 mcg, 400 mcg, 800 mcg, 1.000 mcg, 1200 mcg, 1600 mcg, 2000 mcg, 2400 mcg, 2800 mcg, 3200 mcg, 3600 mcg, and 4000 mcg, although dosage amounts intermediary between any of these specific amounts are also suitable where desired. It should be noted that the above amounts are calculated based on the uncomplexed, non-salt, nonester folate chemical entity. Complexed forms such as glucosaminyl or galactosylamino (or other complexed forms) of the required folates should be present in weight amounts that deliver the stated amounts of the uncomplexed, non-salt, non-ester folate form. Regardless of the form of the particular compounds, a highly preferred dosage form provides 3.83 mg 1-methylfolate, 2.4 mg 1-leucovorin, and 2.5 mg of folic acid.

The B12 component, whether adenosylcobalamin or methylcobalamin are present in amounts which together are at least 10 mcg per dosage form up to 2000 mcg per dosage form, preferably at least 20 mcg. When a salt or ester or amide of these is used, the amount is an amount which provides the stated amount of the non-salt, non-ester, non-amide form. A highly preferred dosage form contains both the adenosylcobalamin and the methylcobalamin (whether in their free form or as a salt or ester or amide of either or each). Preferred dosage amounts of the cobalamin component are a total within a range selected from ranges having a minimum and maximum (with the maximum being greater than the selected minimum) selected from 10 mcg, 20 mcg, 30 mcg, 40 mcg, 50 mcg, 62.5 mcg, 75 mcg, 100 mcg, 125 mcg, 250 mcg, 375 mcg, 500 mcg, 625 mcg, 750 mcg, 875 mcg, 1000 mcg, 1200 mcg, 1250 mcg, 1500 mcg, 1600 mcg, 1750 mcg, 1800 mcg, and 2000 mcg, and highly preferred embodiments have a total of the cobalamin content selected from 10 mcg, 20 mcg, 30 mcg, 40 mcg, 50 mcg, 62.5 mcg, 75 mcg, 100 mcg, 125 mcg, 250 mcg, 375 mcg, 500 mcg, 625 mcg, 750 mcg, 875 mcg, 1000 mcg, 1200 mcg, 1250 mcg, 1500 mcg, 1600 mcg, 1750 mcg, 1800 mcg, and 2000 mcg, each being calculated based on the non-salt, non-ester, non-amide forms thereof, with dosages intermediary to those stated being equally suitable. Highly preferred dosage forms contain a total of 500 mcg and contain both an adenosylcobalamin and a methyl cobalamin. In a most highly preferred form, the dosage form contains 250 mcg of adenosylcobalamin and 250 mcg of methylcobalamin.

Vitamin B16 (as pyridoxyl-5-phosphate), when present, is present in an amount to deliver from 0.125 mg of pyridoxine up to 0.375 mg of pyridoxine, most preferably 0.25 mg of pyridoxine per dosage form with intermediary amounts between those specifically stated being suitable as well.

In addition to the foregoing active agents, the invention formulation can be prepared with a wide range of pharmaceutically acceptable excipients and carriers known in the art, such as binders, disintegrants, dispersants, flow agents, suspending agents, solvents, carrier fluids, flavorings, colorings, buffers, processing aids, etc.

The compositions of the present invention are generally administered once daily, but if desired, a particular daily dose can be administered in fractional doses multiple times a day.

### Example 1

### Formulation A

In this Example, the following formulation is prepared. Amounts are given in mg/dosage unit. Where desired, dosage forms having fractional amounts for administration multiple times per day may also be prepared using proportional amounts of the ingredients.

| Ingredient | |
|---|---|
| Active Components | mg |
| l-methylfolate glucosamine | equivalent to 3.83 mg of l-methylfolate |
| l-leucovorin calcium | equivalent to 2.4 mg of l-leucovorin |
| folic acid | 2.5 mg |
| adenosylcobalamin | 0.25 |
| methylcobalamin | 0.25 |

### Example 2

### Formulation B

In this Example, the following formulation is prepared. Amounts are given in mg/dosage unit. Where desired, dosage forms having fractional amounts for administration multiple times per day may also be prepared using proportional amounts of the ingredients.

| Ingredient | |
|---|---|
| Active Components | mg |
| l-methylfolate glucosamine | equivalent to 3.83 mg of l-methylfolate |
| l-leucovorin calcium | equivalent to 2.4 mg of l-leucovorin |
| folic acid | 2.5 mg |
| vitamin B6 (as pyridoxyl 5′ | 0.25 mg |
| p-hosphate) | |
| adenosylcobalamin | 0.25 mg |
| methylcobalamin | 0.25 mg |

### Example 3

### Use in Depression

The formulations of Examples 1 and 2 are administered to a patient experiencing depression generally once per day. Where the alternate fractional dosage form is used, the dosage form is administered in the appropriate multiple of times per day.

### Example 4

### Use in Improving Mental Alertness

The formulations of Examples 1 and 2 are administered to a patient in need of increasing or improving mental alertness generally once per day. Where the alternate fractional dosage form is used, the dosage form is administered in the appropriate multiple of times per day.

I claim:

1. A folate-B12 composition comprising
(1) at least 3 forms of folate selected from the group comprising
a. at least one of folic acid, a pharmaceutically acceptable salt thereof, a pharmaceutically acceptable ester thereof, and mixtures thereof;
b. at least one of a folinic acid comprising at least 5-formyl-tetrahydofolic acid, a pharmaceutically acceptable salt thereof, a pharmaceutically acceptable ester thereof, and mixtures thereof; and
c. at least one of a 5-methyl-tetrahydrofolic acid, a pharmaceutically acceptable salt thereof, a pharmaceutically acceptable ester thereof, and mixtures thereof; and
(2) at least one vitamin B12 component comprising at least one of
(a) adenosylcobalamine or a pharmaceutically acceptable salt, ester, or amide, thereof or mixtures thereof and
(b) methyl cobalamine or a pharmaceutically acceptable salt, ester, or amide, thereof or mixtures thereof.

2. The composition of claim 1 wherein at least one of said three forms of folate is present as a D-glucosaminyl or D-galactosaminyl form thereof.

3. The composition of claim 1 further comprising one or more additional folate metabolite compounds.

4. The composition of claim 3 wherein said additional folate metabolite compounds are selected from
a. a dihydrofolic acid, a pharmaceutically acceptable salt thereof, a pharmaceutically acceptable ester thereof, and mixtures thereof;
b. a 5,10-methylene-tetrahydrofolic acid, a pharmaceutically acceptable salt thereof, a pharmaceutically acceptable ester thereof, and mixtures thereof; and
c. a tetrahydrofolic acid (other than a 5-methyl-tetrahydrofolic acid and 5,10-methylene-tetrahydrofolic acid), a pharmaceutically acceptable salt thereof, a pharmaceutically acceptable ester thereof, and mixtures thereof.

5. A method of treating a patient in need of metabolically useful folate supplementation comprising administering to said patient the composition of claim 1.

6. The method claim 5, wherein said patient is suffering from depression.

7. A method of prophylactically preventing at least one of the sequella of metabolically useful folate deficiency in a patient at risk thereof comprising administering to said patient the composition of claim 1.

8. The method of claim 7 wherein said sequella of metabolically useful folate deficiency comprises at least depression.

9. A folate-B12 composition comprising at least 3 forms of folate and one reduced form of vitamin B12 selected from the group comprising
1) at least 2 mg of folic acid, a pharmaceutically acceptable salt thereof, a pharmaceutically acceptable ester thereof, and mixtures thereof;
2) at least 2 mg of folinic acid as diasterioisomerically pure (6S) form of 5-formyl-tetrahydrofolic acid, a pharmaceutically acceptable salt thereof, a pharmaceutically acceptable ester thereof, and mixtures thereof; and
3) at least 2 mg of 1-methylfolate as diasterioisomerically pure (6S) form of 5-methyl-tetrahydrofolic acid, a pharmaceutically acceptable salt thereof, a pharmaceutically acceptable ester thereof, and mixtures thereof
4) at least 20 mcg of at least one member selected form adenosylcobalamin and methylcobalamin.

10. A folate-B12 composition consisting essentially of at least 3 forms of folate and at least one reduced form of vitamin B12 selected from the group comprising
1) at least 2 mg of folic acid, a pharmaceutically acceptable salt thereof, a pharmaceutically acceptable ester thereof, and mixtures thereof;
2) at least 2 mg of folinic acid as diasterioisomerically pure (6S) form of 5-formyl-tetrahydrofolic acid, a pharmaceutically acceptable salt thereof, a pharmaceutically acceptable ester thereof, and mixtures thereof; and
3) at least 2 mg of 1-methylfolate as diasterioisomerically pure (6) form of 5-methyl-tetrahydrofolic acid, a pharmaceutically acceptable salt thereof, a pharmaceutically acceptable ester thereof, and mixtures thereof
4) at least 20 mcg of at least one member selected form adenosylcobalamin and methylcobalamin and
5) optionally vitamin B6 as pyridoxyl-5′-phosphate; and
still further optionally containing pharmaceutically acceptable excipients and/or carriers.
